(19) United States
(12) Patent Application Publication
(10) Pub. No.: US 2024/0189200 A1
(43) Pub. Date: Jun. 13, 2024

(54) PROCESS FOR THE COSMETIC TREATMENT OF BRAIDED HAIR WITH A COMPOSITION COMPRISING A THICKENING POLYMER, A DERIVATIVE OF MENTHOL AND A PARTICULAR NON-IONIC SURFACTANT

(71) Applicant: L'Oreal, Paris (FR)

(72) Inventors: Kwezikazi MOLAMODI, WOODMEAD (ZA); Pradisha NAICKER, WOODMEAD (ZA)

(21) Appl. No.: 18/285,074

(22) PCT Filed: Mar. 18, 2022
(86) PCT No.: PCT/EP2022/057152

§ 371 Date: Sep. 29, 2023

(30) Foreign Application Priority Data
Apr. 1, 2021 (ZA) 2021/02218
Apr. 29, 2021 (FR) 2104489

(51) Int. Cl. A61K 8/34; A61K 8/39; A61K 8/49; A61K 8/73; A61K 8/81; A61K 8/92; A61Q 5/00

(52) U.S. Cl. CPC A61K 8/34; A61K 8/39; A61K 8/4993; A61K 8/735; A61K 8/8147; A61K 8/922; A61Q 5/00; A61K 2800/48

(57) ABSTRACT

Process for the cosmetic treatment of braided hair with a composition comprising a thickening polymer, a derivative of menthol and a particular non-ionic surfactant. The present invention relates to a process for the cosmetic treatment of braided hair, utilizing a cosmetic composition comprising in a hydro-alcoholic medium, a thickening polymer, a derivative of menthol and a particular non-ionic surfactant.

The present invention relates to a process for the cosmetic treatment of braided hair, using a composition comprising in a hydro-alcoholic medium, a thickening polymer, a derivative of menthol and a particular non-ionic surfactant.

In some countries, water is rare and it is not possible to wash or shampoo hair as often as wanted. Compositions for cleansing and/or refreshing the scalp and hair have been proposed to consumers, to avoid discomfort and greasy aspect in the period between two shampoos.

In the case of braided hair, it is more complicated to clean and refresh hair, without disrupting the style of braided hair.

One purpose of the present invention is to propose a process for the cosmetic treatment of braided hair, that when applied will give immediate clean and fresh feeling and/or aspect to hair, in particular but not only relief of scratching, tingling and discomfort and will impart a pleasant soothing sensation on scalp, together with a surprising moisturizing effect of hair, while preserving the style of braided hair. Another purpose of the invention is to improve the softness of braided hair.

The Applicant has now discovered that the use of a composition comprising in a hydro-alcoholic medium a thickening polymer, a derivative of menthol and a particular non-ionic surfactant, makes it possible to achieve the objectives outlined above.

Thus, the subject of the invention is a process for the cosmetic treatment of braided hair, comprising a step of applying on said braided hair a cosmetic composition comprising:
(i) one or more thickening polymers,
(ii) one or more compounds of formula (I)

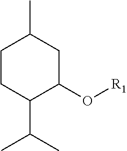

wherein R1 denotes a hydrogen atom or a linear or branched, saturated or unsaturated, C1-C12 alkyl radical, optionally substituted by one or more OH group and/or one or more NRR′ groups with R and R′, identical or different, denoting a hydrogen atom or a linear or branched, saturated or unsaturated, C1-C4 alkyl radical,
(iii) one or more oxyalkylenated C8-C30 fatty alcohols,
(iv) one or more alcohols comprising a C1-C8 alkyl chain, and
(v) water.

It was observed that, surprisingly, the composition used in the present process can improve the softness of braided hair and leads to softened braids.

Advantageously, the composition used in the present invention easily flows to cleanse scalp and hair; it removes odors and dirt from scalp and braided hair, and conducts after application to a fresh and clean sensation on scalp and on braided hair.

The composition does not disrupt the style of braided hair; it shall confer good styling properties, such as a good manageability, to the hair.

Preferably, the composition according to the present invention can be used as a cleansing agent.

It preferably aims at a leave-on application on braided hair; and as the composition after its application on hair, does not need to be rinsed, the process does not require consumption of water and therefore could be considered as a waterless process or a waterless routine for braided hair. Preferably, there is no rinsing step after application of the composition, for example no rinsing step in the 1 hour following the application, preferably in the 2 hours, more preferably in the 12 hours, most preferably in the 24 hours following the application.

In particular, it is a process for improving moisturization and/or softness of braided hair advantageously while preserving the style of braided hair; more particularly, it is a waterless process for improving moisturization and/or softness of braided hair, while preserving the style of braided hair.

Other subjects, characteristics, aspects and advantages of the invention will emerge even more clearly on reading the description and the example that follows.

In that which follows and unless otherwise indicated, the limits of a range of values are included within this range, in particular in the expressions “of between” and “ranging from . . . to . . . ”.

Moreover, the expression “at least one” used in the present description is equivalent to the expression “one or more”.

According to the present invention, the term “braided hair” encompasses natural hair that have been braided.

It also encompasses the style achieved from the process of attaching synthetic fibers to one's natural hair. During this braiding process, synthetic fibers could be weaved together with natural hair to form a two- or three-way braid. Several strands of natural hair could be used to secure each braid. To braid securely, synthetic hair could be bent in half in order to create two sections of the braid entity, a person's own hair then forms the third part of the braid. Braids can also be two-way twists where the natural is wrapped around the synthetic fiber. The synthetic fibers used in protective hairstyles are preferably composed of acrylate polymer-fused with dyes. Other protective styles can be described as plaiting, sewn-in weaves, glue-in weaves and cornrows. Protective hairstyles are usually worn for several weeks and hair is generally washed less frequently.

### Thickening Polymers

The composition used according to the present invention comprises one or more thickening polymer(s).

The term “thickening polymer” means, according to the present invention, a polymer capable, by its presence, of increasing the viscosity of the medium by at least 50 centipoises at 25° C. and at a shear rate of 1 s⁻¹. Preferably, the solution obtained by dissolving the thickening polymer at 1% by weight in water or in a 50/50 by weight water/alcohol mixture exhibits a viscosity at 25° C. and at a shear rate of 1 s⁻¹ which is greater than 100 centipoises. These viscosities can be measured using in particular a viscometer or a rheometer having cone-plate geometry.

Preferably, the thickening polymer(s) are chosen from the thickening polymers comprising acrylic and/or methacrylic units.

The term “polymer comprising acrylic and/or methacrylic units” means, within the meaning of the present invention, a polymer resulting from the polymerization of one or more monomers including one or more monomers of structure:

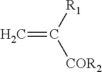

R₁ denoting a hydrogen atom or a linear or branched C₁-C₄ alkyl radical, preferably a hydrogen atom or a methyl radical;
R₂ denoting a hydrogen atom, a hydroxy radical, a linear or branched C₁-C₄ alkyl radical, an NR₃R₄ radical or a linear or branched C₁-C₃₀ alkoxy radical, optionally substituted by one or more hydroxyl radicals or by a quaternary ammonium radical,
R₃ and R₄ denoting a hydrogen atom or an optionally oxyalkylenated C₁-C₃₀ alkyl radical, it being possible for the alkyl radical to comprise a sulfonic group.

As suitable thickening polymers, we can mention:
(a) (meth)acrylic associative thickeners,
(b) cross-linked (meth)acrylic acid homopolymers and copolymers, preferably cross-linked (meth)acrylic acid homopolymers, more preferably cross-linked acrylic acid homopolymers;
(c) copolymers of (meth)acrylic acid and of (C₁-C₆)alkyl (meth)acrylate, preferably cross-linked;
(d) non-ionic homopolymers and copolymers comprising ethylenically unsaturated monomers of ester and/or amide type,
(e) ammonium (meth)acrylate homopolymers or copolymers of ammonium (meth)acrylate and of (meth)acrylamide,
(f) (meth)acrylamido(C₁-C₄)alkylsulfonic acid homopolymers and copolymers,
(g) cross-linked (meth)acryloyloxy(C₁-C₄)alkyltri(C₁-C₄)alkylammonium homopolymers and copolymers, and
(h) mixtures thereof.
(a) The thickening polymers can preferably be chosen from (meth)acrylic associative thickeners, preferably acrylic associative thickeners.
The term “associative thickener” is understood to mean, according to the invention, an amphiphilic thickener comprising both hydrophilic units and hydrophobic units, in particular comprising at least one C₈-C₃₀ fatty chain and at least one hydrophilic unit.
Mention may be made, as acrylic associative thickeners which can be used in the composition according to the invention, of acrylic associative polymers chosen from:
(i) non-ionic amphiphilic polymers comprising at least one fatty chain and at least one hydrophilic unit;
(ii) anionic amphiphilic polymers comprising at least one hydrophilic unit and at least one unit having a fatty chain;
(iii) cationic amphiphilic polymers comprising at least one hydrophilic unit and at least one unit having a fatty chain;
(iv) amphoteric amphiphilic polymers comprising at least one hydrophilic unit and at least one unit having a fatty chain;
   the fatty chains having from 8 to 30 carbon atoms.
(i) The (meth)acrylic associative polymers may preferably be chosen from non-ionic amphiphilic polymers comprising at least one fatty chain and at least one hydrophilic unit; the fatty chains having from 8 to 30 carbon atoms.

These non-ionic amphiphilic polymers can preferably be chosen from:
copolymers of C₁-C₆ alkyl methacrylates or acrylates and of amphiphilic monomers comprising at least one fatty chain (for example oxyethylenated (C₈-C₂₂)alkyl acrylates), such as, for example, the oxyethylenated methyl methacrylate/stearyl acrylate copolymer sold by Goldschmidt under the name Antil 208; and
copolymers of hydrophilic methacrylates or acrylates and of hydrophobic monomers comprising at least one fatty chain (for example (C₈-C22)alkyl (meth)acrylates), such as, for example, polyethylene glycol methacrylate/lauryl methacrylate copolymer.
(ii) The (meth)acrylic associative polymers may preferably be chosen from anionic amphiphilic polymers comprising at least one hydrophilic unit and at least one unit having a fatty chain; the fatty chains having from 8 to 30 carbon atoms.
These anionic amphiphilic polymers can be chosen from those comprising at least one hydrophilic unit of unsaturated olefinic carboxylic acid type and at least one hydrophobic unit of unsaturated carboxylic acid (C₁₀-C₃₀)alkyl ester type. They are preferably chosen from those for which the hydrophilic unit of unsaturated olefinic carboxylic acid type corresponds to the monomer of following formula (III):

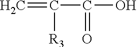

in which, R₃ denotes H or CH₃ or C₂H₅, that is to say acrylic acid, methacrylic acid or ethacrylic acid units, and for which the hydrophobic unit of unsaturated carboxylic acid (C₁₀-C₃₀)alkyl ester type corresponds to the monomer of following formula (IV):

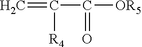

in which, R₄ denotes H or CH₃ or C₂H₅ (that is to say acrylate, methacrylate or ethacrylate units) and preferably H (acrylate units) or CH₃ (methacrylate units), R₅ denoting a C₁₀-C₃₀ and preferably C₁₂-C₂₂ alkyl radical.
(C₁₀-C₃₀)alkyl esters of unsaturated carboxylic acids in accordance with the invention comprise, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic amphiphilic polymers of this type are, for example, described and prepared according to U.S. Pat. Nos. 3,915,921 and 4,509,949.

The anionic amphiphilic polymers which can be used in the present invention can more particularly denote polymers formed from a mixture of monomers comprising:
acrylic acid and one or more esters of following formula (V):

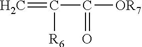

in which, R₆ denotes H or CH₃, R₇ denoting an alkyl radical having from 12 to 22 carbon atoms, and a crosslinking agent, such as, for example, those constituted of from 95% to 60% by weight of acrylic acid (hydrophilic unit), 4% to 40% by weight of C₁₀-C₃₀ alkyl acrylate (hydrophobic unit) and 0% to 6% by weight of crosslinking polymerizable monomer, or 98% to 96% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer, and
essentially acrylic acid and lauryl methacrylate, such as the product formed from 66% by weight of acrylic acid and 34% by weight of lauryl methacrylate.

The said crosslinking agent is for example a monomer comprising a group

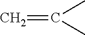

with at least one other polymerizable group, the unsaturated bonds of which are non-conjugated with respect to one another. Mention may in particular be made of polyallyl ethers, such as, in particular, polyallyl sucrose and polyallyl pentaerythritol.

Among the said polymers above, preference is given, according to the present invention, to the products sold by Goodrich under the trade names Pemulen TR1, Pemulen TR2 and Carbopol 1382, and more preferably still Pemulen TR1, and to the product sold by S.E.P.C. under the name Coatex SX.

Mention may also be made, as anionic amphiphilic polymers having fatty chains, of the copolymer of methacrylic acid, methyl acrylate and dimethyl-meta-isopropenylbenzyl isocyanate of ethoxylated alcohol sold under the name Viscophobe DB 1000 by Amerchol.

Mention may be made, as other anionic amphiphilic polymers having fatty chains, of those comprising at least one acrylic monomer having sulfonic group(s), in free or partially or completely neutralized form, and comprising at least one hydrophobic portion.

The hydrophobic portion present in the polymers of the invention preferably comprises from 8 to 22 carbon atoms, more preferably still from 8 to 18 carbon atoms and more particularly from 12 to 18 carbon atoms.

Preferably, the sulfonic polymers in accordance with the invention are partially or completely neutralized by an inorganic base (sodium hydroxide, potassium hydroxide or aqueous ammonia) or an organic base, such as mono-, di- or triethanolamine, an aminomethylpropanediol, N-methylglucamine, basic amino acids, such as arginine and lysine, and mixtures of these compounds.

The sulfonic amphiphilic polymers in accordance with the present invention generally have a number-average molecular weight ranging from 1000 to 20 000 000 g/mol, preferably ranging from 20 000 to 5 000 000 g/mol and more preferably still ranging from 100 000 to 1 500 000 g/mol.

The sulfonic amphiphilic polymers according to the invention may or may not be cross-linked. Crosslinked amphiphilic polymers are preferably chosen.

When they are cross-linked, the crosslinking agents can be chosen from polyolefinically unsaturated compounds commonly used for the crosslinking of polymers obtained by radical polymerization. Mention may be made, for example, of divinylbenzene, diallyl ether, dipropylene glycol diallyl ether, polyglycol diallyl ethers, triethylene glycol divinyl ether, hydroquinone diallyl ether, ethylene glycol di(meth)acrylate or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allyl ethers of alcohols of the sugar series, or other allyl or vinyl ethers of polyfunctional alcohols, and also allyl esters of phosphoric and/or vinylphosphonic acid derivatives, or mixtures of these compounds.

Methylenebisacrylamide, allyl methacrylate or trimethylolpropane triacrylate (TMPTA) will more particularly be used. The degree of crosslinking will generally vary from 0.01 mol % to 10 mol % and more particularly from 0.2 mol % to 2 mol %, with respect to the polymer.

The acrylic monomers having sulfonic group(s) are chosen in particular from (meth)acrylamido(C₁-C₂₂)alkylsulfonic acids and N—(C₁-C₂₂)alkyl(meth)acrylamido(C₁-C₂₂)alkylsulfonic acids, such as undecylacrylamidomethanesulfonic acid, and also their partially or completely neutralized forms.

More preferably, use will be made of (meth)acrylamido(C₁-C₂₂)alkylsulfonic acids, such as, for example, acrylamidomethanesulfonic acid, acrylamidoethanesulfonic acid, acrylamidopropanesulfonic acid, 2-acrylamido-2-methylpropanesulfonic acid, methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid or 2-acrylamido-2,6-dimethyl-3-heptanesulfonic acid, and also their partially or completely neutralized forms.

More particularly, use can be made of 2-acrylamido-2-methylpropanesulfonic acid (AMPS®), and also its partially or completely neutralized forms.

The amphiphilic polymers in accordance with the invention can in particular be chosen from random amphiphilic AMPS® polymers modified by reaction with an n-mono(C₆-C₂₂)alkylamine or a di[n-(C₆-C₂₂)alkyl]amine, such as those described in Patent Application WO 00/31154; the polymers described in this patent application form part of the content of the present description. These polymers can also comprise other ethylenically unsaturated hydrophilic monomers chosen, for example, from (meth)acrylic acids, their β-substituted alkyl derivatives or their esters obtained with monoalcohols or mono- or polyalkylene glycols, (meth)acrylamides, vinylpyrrolidone, maleic anhydride, itaconic acid or maleic acid, or mixtures of these compounds.

The polymers of the invention can be chosen from amphiphilic copolymers of AMPS® and of at least one ethylenically unsaturated hydrophobic monomer comprising at least one hydrophobic portion having from 8 to 50 carbon atoms, more preferably from 8 to 22 carbon atoms, more preferably still from 8 to 18 carbon atoms and more particularly from 12 to 18 carbon atoms.

These same copolymers can additionally comprise one or more ethylenically unsaturated monomers not comprising a fatty chain, such as (meth)acrylic acids, their β-substituted alkyl derivatives or their esters obtained with monoalcohols or mono- or polyalkylene glycols, (meth)acrylamides, vinylpyrrolidone, maleic anhydride, itaconic acid or maleic acid, or mixtures of these compounds.

These copolymers are described in particular in Patent Application EP-A-750 899, in U.S. Pat. No. 5,089,578 and in the following Yotaro Morishima publications:
“Self-assembling amphiphilic polyelectrolytes and their nanostructures”, Chinese Journal of Polymer Science, Vol. 18, No. 40, (2000), 323-336;
“Micelle formation of random copolymers of sodium 2-(acrylamido)-2-methylpropanesulfonate and a non-ionic surfactant macromonomer in water as studied by fluorescence and dynamic light scattering”, Macromolecules, 2000, Vol. 33, No. 10, 3694-3704;
“Solution properties of micelle networks formed by non-ionic moieties covalently bound to a polyelectrolyte: salt effects on rheological behavior”, Langmuir, 2000, Vol. 16, No. 12, 5324-5332;
“Stimuli responsive amphiphilic copolymers of sodium 2-(acrylamido)-2-methylpropanesulfonate and associative macromonomers”, Polym. Preprint, Div. Polym. Chem., 1999, 40(2), 220-221.

The ethylenically unsaturated hydrophobic monomers of these specific copolymers are preferably chosen from the acrylates or acrylamides of following formula (VI):

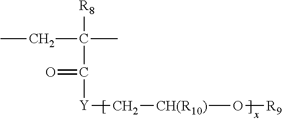

in which, R₈ and R₁₀, which are identical or different, denote a hydrogen atom or a linear or branched C₁-C₆ alkyl radical (preferably methyl); Y denotes O or NH; R₉ denotes a hydrophobic hydrocarbon radical comprising at least from 8 to 50 carbon atoms, more preferably from 8 to 22 carbon atoms, more preferably still from 6 to 18 carbon atoms and more particularly from 12 to 18 carbon atoms; and x denotes a number of moles of alkylene oxide and varies from 0 to 100.

The R₉ radical is preferably chosen from linear C₆-C₁₈ (for example n-hexyl, n-octyl, n-decyl, n-hexadecyl or n-dodecyl) alkyl radicals or branched or cyclic (for example cyclododecane (C₁₂) or adamantane (C₁₀)) alkyl radicals; C₆-C₁₈ perfluoroalkyl radicals (for example the group of formula —(CH₂)₂—(CF₂)₉—CF₃); the cholesteryl (C₂₇) radical or a cholesterol ester residue, such as the cholesteryl oxyhexanoate group; or polycyclic aromatic groups, such as naphthalene or pyrene. Preference is more particularly given, among these radicals, to linear alkyl radicals and more particularly to the n-dodecyl radical.

According to a particularly preferred form of the invention, the monomer of formula (VI) comprises at least one alkylene oxide unit (x≥1) and preferably a polyoxyalkylene chain. The polyoxyalkylene chain preferably consists of ethylene oxide units and/or of propylene oxide units and more particularly still consists of ethylene oxide units. The number of oxyalkylene units generally varies from 3 to 100, more preferably from 3 to 50 and more preferably still from 7 to 25.

Mention may be made, among these polymers, of:
copolymers which are or are not cross-linked and which are or are not neutralized, comprising from 15% to 60% by weight of AMPS® units and from 40% to 85% by weight of (C₈-C₁₆)alkyl(meth)acrylamide units or of C₈-C₁₆)alkyl (meth)acrylate units, with respect to the polymer, such as those described in Application EP-A-750 899; and
terpolymers comprising from 10 mol % to 90 mol % of acrylamide units, from 0.1 mol % to 10 mol % of AMPS® units and from 5 mol % to 80 mol % of n-(C₆-C₁₈)alkylacrylamide units, such as those described in U.S. Pat. No. 5,089,578.

Mention may also be made of copolymers of completely neutralized AMPS® and of dodecyl methacrylate, and also non-cross-linked and cross-linked copolymers of AMPS® and of n-dodecylmethacrylamide, such as those described in the above-mentioned papers by Morishima.

Mention will more particularly be made of the copolymers constituted of 2-acrylamido-2-methylpropanesulfonic acid (AMPS®) units of following formula (VII):

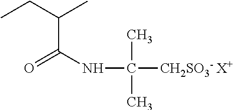

in which, X⁺ is a proton, an alkali metal cation, an alkaline earth metal cation or the ammonium ion,
and of units of following formula (VIII):

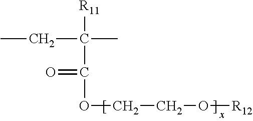

in which, X⁺ denotes an integer varying from 3 to 100, preferably from 5 to 80 and more preferably from 7 to 25, R₁₁ has the same meaning as that indicated above for R₈ in the formula (VI) and R₁₂ denotes a linear or branched C₆-C₂₂ and more preferably C₁₀-C₂₂ alkyl.

The polymers particularly preferred are those for which x=25, R₁₁ denotes methyl and R₁₂ represents n-dodecyl.

The polymers for which X⁺ denotes sodium or ammonium are more particularly preferred.
(iii) The (meth)acrylic associative thickeners may preferably be chosen from cationic amphiphilic polymers comprising at least one hydrophilic unit and at least one unit having a fatty chain; the fatty chains having from 10 to 30 carbon atoms.
The cationic amphiphilic polymers are preferably chosen from polyacrylates having aminated side groups.
The polyacrylates having quaternized or non-quaternized aminated side groups possess, for example, hydrophobic groups of the steareth-20 (polyoxyethylenated (20) stearyl alcohol) or (C₁₀-C₃₀)alkyl PEG-20 itaconate type.
Mention may be made, as examples of polyacrylates having aminated side chains, of the polymers 8781-124B or 9492-103 or Structure Plus from National Starch.
(iv) The (meth)acrylic associative polymers may preferably be chosen from amphoteric amphiphilic polymers comprising at least one hydrophilic unit and at least one unit having a fatty chain; the fatty chains having from 10 to 30 carbon atoms.
Among these amphoteric amphiphilic polymers, mention may be made of methacrylamidopropyltrimethylammonium chloride/acrylic acid/C₁₀-C₃₀ alkyl methacrylate copolymers, the alkyl radical preferably being a stearyl radical.
(b) The thickening polymers can preferably be chosen from cross-linked (meth)acrylic acid homopolymers and copolymers, preferably cross-linked (meth)acrylic acid homopolymers, more preferably cross-linked acrylic acid homopolymers.
Among the cross-linked (meth)acrylic acid homopolymers, mention may be made of those cross-linked by an allyl ether of an alcohol of the sugar series, such as, for example, the products sold under the names Carbopol, 910, 934, 940, 941, 934 P, 980, 981, 2984, 5984 and Carbopol Ultrez 10 Polymer by Lubrizol or the products sold under the names Synthalen M and Synthalen K by 3 VSA, Cosmedia SP®; mention may also be made of cross-linked sodium polyacrylate such as those comprising 90% of dry matter and 10% of water, Cosmedia SPL® or sodium polyacrylate as inverse emulsion comprising approximately 60% of dry active matter, an oil (hydrogenated polydecene) and a surfactant (PPG-5 Laureth-5), both sold by Cognis, or partially neutralized cross-linked sodium polyacrylates occurring in the form of an inverse emulsion comprising at least one polar oil, for example that sold under the name Luvigel® EM by BASF.
(c) The thickening polymers can preferably be chosen from copolymers of (meth)acrylic acid and of (C₁-C₆)alkyl acrylate, preferably cross-linked copolymers of (meth)acrylic acid and of (C1-C6)alkyl acrylate.
Among these copolymers of (meth)acrylic acid and of (C₁-C₆)alkyl acrylate, mention may be made of cross-linked copolymers of (meth)acrylic acid and of ethyl acrylate, such as the product sold under the name Viscoatex 538C by Coatex, which is a cross-linked copolymer of methacrylic acid and of ethyl acrylate as an aqueous dispersion comprising 38% of active material, or the product sold under the name Aculyn 33 by Rohm & Haas, which is a cross-linked copolymer of acrylic acid and of ethyl acrylate as an aqueous dispersion comprising 28% of active material. Mention may more particularly be made of the cross-linked methacrylic acid/ethyl acrylate copolymer in the form of an aqueous 30% dispersion manufactured and sold under the name Carbopol Aqua SF-1 by Noveon.
(d) The thickening polymers can preferably be chosen from non-ionic homopolymers and copolymers comprising ethylenically unsaturated monomers of ester and/or amide type.
Among these non-ionic homopolymers or copolymers comprising ethylenically unsaturated monomers of ester and/or amide type, mention may be made of the products sold under the names of: Cyanamer P250 by Cytec (polyacrylamide); PMMA MBX-8C by US Cosmetics (methyl methacrylate/ethylene glycol dimethacrylate copolymer); Acryloid B66 by Rohm & Haas (butyl methacrylate/methyl methacrylate copolymer); or BPA 500 by Kobo (polymethyl methacrylate).
(e) The thickening polymers can preferably be chosen from ammonium acrylate homopolymers or copolymers of ammonium acrylate and of acrylamide.
Among ammonium acrylate homopolymers, mention may be made of the product sold under the name Microsap PAS 5193 by Hoechst.
Among copolymers of ammonium acrylate and of acrylamide, mention may be made of the product sold under the name Bozepol C Nouveau or the product PAS 5193 sold by Hoechst (they are described and prepared in the documents FR-2 416 723, U.S. Pat. Nos. 2,798,053 and 2,923,692).
(f) The thickening polymers can preferably be chosen from (meth)acrylamido(C₁-C₄)alkylsulfonic acid homopolymers and copolymers.
Among these (meth)acrylamido(C₁-C₄)alkylsulfonic acid homopolymers and copolymers, preference is preferably given to the use of cross-linked polymers.
More particularly still, they are partially or completely neutralized.

These are water-soluble or water-swellable polymers.
Mention may be made, among these polymers, of:
polyacrylamidomethanesulfonic acid,
polyacrylamidoethanesulfonic acid,
polyacrylamidopropanesulfonic acid,
poly(2-acrylamido-2-methylpropanesulfonic acid),
poly(2-methacrylamido-2-methylpropanesulfonic acid),
poly(2-acrylamido-n-butanesulfonic acid).
Polymers of this type and in particular cross-linked and partially or completely neutralized poly(2-acrylamido-2-methylpropanesulfonic acids) are known, described and prepared in Patent Application DE-196 25 810.
They are generally characterized in that they comprise, randomly distributed from 90% to 99.9% by weight of units of following formula (IX):

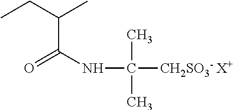

in which, X⁺ denotes a cation or a mixture of cations, including H⁺, and from 0.01% to 10% by weight of at least one crosslinking unit having at least two olefinic double bonds; the proportions by weight being defined with respect to the total weight of the polymer.
X⁺ represents a cation or a mixture of cations chosen in particular from a proton, an alkali metal cation, a cation equivalent to that of an alkaline earth metal, or the ammonium ion.
Preferably, the cross-linked and neutralized poly(2-acrylamido-2-methylpropanesulfonic acid) comprises from 98% to 99.5% by weight of units of formula (IX) and from 0.5% to 2% by weight of crosslinking units.
The crosslinking units having at least two olefinic double bonds are chosen, for example, from dipropylene glycol diallyl ether, polyglycol diallyl ethers, triethylene glycol divinyl ether, hydroquinone diallyl ether, tetraallyloxyethane or other polyfunctional alcohol allyl or vinyl ethers, tetraethylene glycol diacrylate, triallylamine, trimethylolpropane diallyl ether, methylenebisacrylamide or divinylbenzene.

The crosslinking units having at least two olefinic double bonds are more particularly still chosen from those corresponding to the following general formula (X):

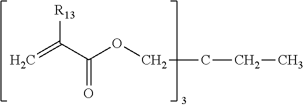

in which, R₁₃ denotes a hydrogen atom or a C₁-C₄ alkyl and more particularly methyl (trimethylolpropane triacrylate).
The acrylamido-2-methylpropanesulfonic acid (AMPS®) polymers according to the present invention generally have a number-average molecular weight ranging from ranging from 50 000 to 10 000 000 g/mol, and preferably ranging from 80 000 to 8 000 000 g/mol.
Among these AMPS® polymers, mention may preferably be made of:
cross-linked sodium acrylamido-2-methyl propane sulfonate polymers, such as for example the product sold under the name Simulgel 800 (INCI name: Sodium Polyacryloyldimethyl Taurate), cross-linked ammonium acrylamido-2-methyl propane sulfonate polymers, such as for example the polymers described in the patent EP 0 815 928 B1, or the product sold under the trade name Hostacerin AMPS® by Clariant (INCI name: Ammonium Polydimethyltauramide);
AMPS/acrylamide copolymers, in particular cross-linked acrylamide/sodium acrylamido-2-methyl propane sulfonate copolymers such as for example the product sold under the name SEPIGEL 305 (INCI name: Polyacrylamide/C₁₃-C₁₄ Isoparaffin/Laureth-7) or the product sold under the name Simulgel 600 by Seppic (INCI name: Acrylamide/Sodium acryloyldimethyltaurate/Isohexadecane/Polysorbate-80);
AMPS®/vinylpyrrolidone copolymers or AMPS®/vinylformamide copolymers, such as for example the copolymer used in the product sold under the name Aristoflex AVC® by Clariant (INCI name: Ammonium Acryloyldiméthyltaurate/VP Copolymer) neutralized by sodium carbonate or potassium carbonate;
AMPS®/sodium acrylate copolymers, such as for example the AMPS/sodium acrylate copolymer used in the product sold under the name Simulgel EG® by Seppic or under the name Sepinov EM (INCI name: Hydroxyethyl Acrylate/Sodium Acryloyldimethyl taurate copolymer);
AMPS®/hydroxyethyl acrylate copolymers, such as for example the AMPS®/hydroxyéthyl acrylate copolymère used in the product sold under the name Simulgel NS® by Seppic (INCI name: Hydroxyethyl Acrylate/Sodium Acryloyldimethyltaurate copolymer (And) Squalane (And) Polysorbate 60) or the sodium acrylamido-2-methyl propane sulfonate/Hydroxyethylacrylate copolymer sold under the name Sepinov EMT 10 (INCI name: Hydroxyethyl Acrylate/Sodium Acryloyldimethyl taurate copolymer).
(g) The thickening polymers can preferably be chosen from cross-linked methacryloyloxy(C₁-C₄)alkyltri(C₁-C₄)alkylammonium homopolymers and copolymers.
Among these cross-linked methacryloyloxy(C₁-C₄)alkyltri(C₁-C₄)alkylammonium homopolymers and copolymers, mention may be made of cross-linked polymers of methacryloyloxy(C₁-C₄)alkyltri(C₁-C₄)alkylammonium salts, such as the polymers obtained by homopolymerization of dimethylaminoethyl methacrylate quaternized by methyl chloride or by copolymerization of acrylamide with dimethylaminoethyl methacrylate quaternized by methyl chloride, the homo- or copolymerization being followed by crosslinking by an olefinically unsaturated compound, in particular methylenebisacrylamide.
Use may more particularly be made of a cross-linked acrylamide/methacryloyloxyethyltrimethylammonium chloride (20/80 by weight) copolymer in the form of a dispersion comprising 50% by weight of the said copolymer in mineral oil. This dispersion is sold under the name of Salcare® SC 92 by Ciba. Use may also be made of a cross-linked homopolymer of methacryloyloxyethyltrimethylammonium chloride comprising approximately 50% by weight of the homopolymer in mineral oil or in a liquid ester. These dispersions are sold under the names of Salcare® SC 95 and Salcare® SC 96 by Ciba.

Preferably, the thickening polymer(s) is/are chosen from cross-linked (meth)acrylic acid homopolymers, preferably cross-linked by an allyl ether of an alcohol of the sugar series; and/or cross-linked copolymers of (meth)acrylic acid and of (C1-C6)alkyl acrylate; preferably cross-linked copolymers of (meth)acrylic acid and of ethyl acrylate.

The total amount of thickening polymer(s) present in the composition according to the present invention preferably ranges from 0.01 to 5% by weight, more preferably from 0.02 to 1% by weight, and better still from 0.05 to 0.5% by weight, relative to the total weight of the composition.

Preferably, the total amount of thickening polymer(s) chosen from cross-linked (meth)acrylic acid homopolymers, preferably cross-linked by an allyl ether of an alcohol of the sugar series; and/or cross-linked copolymers of (meth)acrylic acid and of (C1-C6)alkyl acrylate; preferably cross-linked copolymers of (meth)acrylic acid and of ethyl acrylate, present in the composition according to the present invention ranges from 0.01 to 5% by weight, more preferably from 0.02 to 1% by weight, and better still from 0.05 to 0.5% by weight, relative to the total weight of the composition.

### Compounds of Formula (I)

The composition used according to the invention also comprises one or more compound(s) of formula (I)

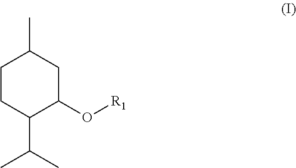

wherein, R₁ denotes a hydrogen atom or a linear or branched, saturated or unsaturated, C₁-C₁₂ alkyl radical, optionally substituted by one or more hydroxyl (OH) groups and/or one or more NRR′ groups with R and R′, identical or different, denoting a hydrogen atom or a linear or branched, saturated or unsaturated, C₁-C₄ alkyl radical.
Preferably, R₁ denotes a hydrogen atom or a linear or branched, saturated or unsaturated, C₁-C₆ alkyl radical, substituted by one or more hydroxyl groups. More preferably, R1 denotes a linear and saturated C₁-C₆ alkyl radical, substituted by one or more hydroxyl groups, and better still by one or two hydroxyl groups.
Advantageously, the compound(s) of formula (I) is/are chosen from menthol, menthoxy propanediol, and mixture thereof.

The total amount of compound(s) of formula (I) present in the composition according to the present invention preferably ranges from 0.05 to 5% by weight, and more preferably from 0.1 to 1% by weight, relative to the total weight of the composition. Preferably, the total amount of menthol, menthoxy propanediol, and mixture thereof, present in the composition according to the present invention ranges from 0.05 to 5% by weight, and more preferably from 0.1 to 1% by weight, relative to the total weight of the composition.

### Non-ionic Surfactants

The composition used according to the invention comprises one or more oxyalkylenated C₈-C₃₀ fatty alcohols, preferably one or more oxyethylenated C₈-C₃₀ fatty alcohols. These compounds are non-ionic surfactants.

The term “fatty alcohol”, according to the present invention, means an alcohol comprising linear or branched, saturated or unsaturated alkyl chain comprising at least 8 carbon atoms.

According to the present invention, an oxyalkylenated fatty alcohol means a fatty alcohol comprising at least one oxyalkylenated (alkylene oxide) unit, and an oxyethylenated fatty alcohol means a fatty alcohol comprising at least one oxyethylenated (ethylene oxide) unit.

According to the present invention, such a fatty alcohol comprises from 8 to 30 carbon atoms, more preferably from 10 to 24 carbon atoms and better still from 10 to 18 carbon atoms.

Preferably, the fatty alcohols according to the present invention comprise a linear and saturated alkyl chain.

The oxyalkylene units are more particularly oxyethylene or oxypropylene units, or a combination thereof, preferably oxyethylene units.

The number of oxyalkylene units, and particularly oxyethylene units, advantageously ranges from 1 to 50, preferably from 2 to 30, more preferably from 5 to 20, and better still from 10 to 15.

The oxyalkylenated C₈-C₃₀ fatty alcohol(s) used in the present invention is/are preferably chosen from oxyethylenated fatty alcohols comprising a C₁₀-C₁₈ alkyl chain and from 5 to 20 oxyethylene units, and more preferably from oxyethylenated fatty alcohols comprising a C₁₀-C₁₈ alkyl chain and from 10 to 15 of oxyethylene units.

More preferably, the oxyalkylenated fatty alcohol is the oxyethylenated lauryl alcohol containing 12 units of ethylene oxide (laureth-12).

The total amount of oxyalkylenated C₈-C₃₀ fatty alcohols present in the composition according to the present invention preferably ranges from 0.1 to 10% by weight, preferably from 0.2 to 5% by weight, more preferably from 0.5 to 3% by weight, relative to the total weight of the composition.

Preferably, the total amount of oxyethylenated fatty alcohols comprising a C10-C18 alkyl chain and from 5 to 20 oxyethylene units, and more preferably from oxyethylenated fatty alcohols comprising a C10-C18 alkyl chain and from 10 to 15 of oxyethylene units, present in the composition according to the present invention ranges from 0.1 to 10% by weight, preferably from 0.2 to 5% by weight, more preferably from 0.5 to 3% by weight, relative to the total weight of the composition.

The composition according to the present invention may further comprise one or more additional non-ionic surfactant(s) different from the above-described oxyalkylenated C₈-C₃₀ fatty alcohols.

The non-ionic surfactant(s) that may be used in the compositions are described, for example, in the Handbook of Surfactants by M. R. Porter, published by Blackie & Son (Glasgow and London), 1991, pp. 116-178.

Examples of additional non-ionic surfactants that may be mentioned include the following non-ionic surfactants:
oxyalkylenated (C₈-C₂₄)alkylphenols;
saturated or unsaturated, linear or branched, glycerolated C₈-C₃₀ alcohols, comprising one or two fatty chains;
saturated or unsaturated, linear or branched, oxyalkylenated C₈-C₃₀ fatty acid amides;
esters of saturated or unsaturated, linear or branched, C₈-C₃₀ acids and of polyethylene glycols;
esters of saturated or unsaturated, linear or branched, C₈-C₃₀ acids and of sorbitol, preferably oxyethylenated;
fatty acid esters of sucrose;
(C₈-C₃₀)alkyl(poly)glucosides, (C₈-C₃₀)alkenyl(poly) glucosides, which are optionally oxyalkylenated (0 to 10 oxyalkylene units) and comprising from 1 to 15 glucose units, (C₈-C₃₀)alkyl (poly)glucoside esters;
saturated or unsaturated, oxyethylenated plant oils;
condensates of ethylene oxide and/or of propylene oxide, alone or as mixtures;
N—(C₈-C₃₀)alkylglucamine and N—(C₈-C₃₀) acylmethylglucamine derivatives;
aldobionamides;
amine oxides;
and mixtures thereof.

The oxyalkylene units are more particularly oxyethylene or oxypropylene units, or a combination thereof, preferably oxyethylene units.

The number of moles of ethylene oxide and/or propylene oxide preferably ranges from 1 to 250, more particularly from 2 to 100 and better still from 2 to 50; the number of moles of glycerol ranges especially from 1 to 50 and better still from 1 to 10.

Advantageously, the additional non-ionic surfactants according to the invention do not comprise any oxypropylene units.

As examples of glycerolated nonionic surfactants, use is preferably made of monoglycerolated or polyglycerolated C₈-C₄₀ alcohols, comprising from 1 to 50 mol of glycerol and preferably from 1 to 10 mol of glycerol.

Examples of saturated or unsaturated, oxyethylenated plant oils that may be mentioned include derivatives of polyoxyethyleneglycol hydrogenated vegetable/plant oil. Preferably, the number of polyoxyethyleneglycol groups ranges from 1 to 250, more preferably from 2-100, better still from 1 to 60, and even more preferably from 50 to 60.

The hydrogenated plant oil component may or may not contain linear or branched, saturated or unsaturated C₈-C₁₈ carbon chains substituted by one or more ethyleneglycol groups.

The additional non-ionic surfactant(s), different from the non-ionic surfactant(s) chosen from oxyalkylenated C₈-C₃₀ fatty alcohols, is (are) advantageously chosen from esters of saturated or unsaturated, linear or branched, C₈-C₃₀ acids and of sorbitol, preferably oxyethylenated; saturated or unsaturated, oxyethylenated plant oils, and mixtures thereof.

Preferably, the additional non-ionic surfactant(s), different from oxyalkylenated C₈-C₃₀ fatty alcohols, is/are chosen from
saturated or unsaturated, oxyethylenated plant oils, more preferably from hydrogenated 50-60 oxyethylenated (i.e. 50-60 OE units) plant oils, better still from hydrogenated 50-60 oxyethylenated castor oils, and even more preferably said additional non-ionic surfactant is PEG 60 hydrogenated castor oil;
esters of saturated or unsaturated, linear or branched, C8-C30 acids and of sorbitol, preferably oxyethylenated with 1 to 18 OE; more preferably chosen from saturated or unsaturated, linear C8-C30 acids and of sorbitol, oxyethylenated preferably with 2 to 10 OE; and better still from saturated or unsaturated, linear C10-C24 acids and of sorbitol, oxyethylenated with 2 to 10 OE. An example that may be mentioned is sorbitan monolaurate with 4 OE units (Polysorbate 21);
and mixtures thereof.

More preferably, the composition used according to the invention comprises:
one or more saturated or unsaturated, oxyethylenated plant oils, more preferably from hydrogenated 50-60 oxyethylenated (i.e. 50-60 OE units) plant oils, better still from hydrogenated 50-60 oxyethylenated castor oils, and even more preferably said additional non-ionic surfactant is PEG 60 hydrogenated castor oil; and
one or more esters of saturated or unsaturated, linear or branched, C8-C30 acids and of sorbitol, preferably oxyethylenated with 1 to 18 OE; more preferably chosen from saturated or unsaturated, linear C8-C30 acids and of sorbitol, oxyethylenated preferably with 2 to 10 OE; and better still from saturated or unsaturated, linear C10-C24 acids and of sorbitol, oxyethylenated with 2 to 10 OE. An example that may be mentioned is sorbitan monolaurate with 4 OE units (Polysorbate 21).

Much more preferably, the composition used according to the invention comprises PEG-60 hydrogenated castor oil and sorbitan monolaurate with 4 OE units (polysorbate 21).

The total amount of the additional non-ionic surfactant(s) different from oxyalkylenated C₈-C₃₀ fatty alcohols, when present, preferably ranges from 0.05 to 5% by weight, preferably from 0.1 to 4% by weight, and more preferably from 0.2 to 3% by weight, relative to the total weight of the composition.

The total amount of additional non-ionic surfactant(s) chosen from saturated or unsaturated, oxyethylenated plant oils, when present, preferably ranges from 0.05 to 1% by weight, more preferably of from 0.1 to 0.5% by weight, and better still from 0.15 to 0.35% by weight, relative to the total weight of the composition.

The total amount of additional non-ionic surfactant(s) chosen from esters of saturated or unsaturated, linear or branched, C8-C30 acids and of sorbitol, preferably oxyethylenated with 1 to 18 OE, when present, preferably ranges from 0.05 to 5% by weight, preferably from 0.1 to 2, and more preferably from 0.2 to 1% by weight, relative to the total weight of the composition.

Preferably, when additional non-ionic surfactants are present in the composition of the invention, their total amount is lower than the total amount of oxyalkylenated C₈-C₃₀ fatty alcohols.

In other words, the weight ratio between the total amount of additional non-ionic surfactant(s) and the total amount of non-ionic surfactant(s) chosen from oxyalkylenated C₈-C₃₀ fatty alcohols is preferably lower than 1.

### Water-Soluble Organic Solvent

The composition according to the present invention also comprises one or more alcohols comprising a C₁-C₈ alkyl chain.

According to the present invention, the alcohols comprising a C1-C8 alkyl chain are water-soluble solvents and have a solubility of greater than or equal to 5% by weight in water at 25° C. and at atmospheric pressure (1 atm).

The alcohols comprising a C1-C8 alkyl chain preferably comprise from 1 to 8, preferably from 1 to 3, OH groups and from 2 to 8, preferably from 2 to 6, carbon atoms. The alcohols comprising a C1-C8 alkyl chain are preferably chosen from linear or branched, preferably saturated, monoalcohols or diols comprising from 2 to 8 carbon atoms, such as ethyl alcohol, isopropyl alcohol, hexylene glycol (2-methyl-2,4-pentanediol), neopentyl glycol, 3-methyl-1,5-pentanediol, butylene glycol, dipropylene glycol or propylene glycol; aromatic alcohols, such as phenylethyl alcohol; polyols comprising more than two hydroxyl functional groups, such as glycerol; polyol ethers, such as, for example, ethylene glycol monomethyl, monoethyl and mono-butyl ethers, propylene glycol or its ethers, such as, for example, propylene glycol monomethyl ether; and also diethylene glycol alkyl ethers, in particular C₁-C₄ alkyl ethers, such as, for example, diethylene glycol monoethyl ether or monobutyl ether, alone or as a mixture.

Preferably, said alcohols comprising a C1-C8 alkyl chain is/are chosen from mono-alcohols comprising from 2 to 8 carbon atoms, diols comprising from 2 to 8 carbon atoms and mixtures thereof, and more preferably from monoalcohols comprising from 2 to 8 carbon atoms, ethanol being most preferred.

The total amount of alcohol(s) comprising a C₁-C₈ alkyl chain present in the composition according to the invention preferably ranges from 10 to 50% by weight, more preferably from 15 to 40% by weight, most preferably from 18 to 35% by weight, relative to the total weight of the composition.

Preferably, the total amount of monoalcohols comprising from 2 to 8 carbon atoms, diols comprising from 2 to 8 carbon atoms and mixtures thereof, and more preferably from monoalcohols comprising from 2 to 8 carbon atoms, present in the composition according to the invention ranges from 10 to 50% by weight, more preferably from 15 to 40% by weight, most preferably from 18 to 35% by weight, relative to the total weight of the composition.

Preferably, the weight ratio between the total amount of compound(s) of formula (I) and the total amount of alcohols comprising a C1-C8 alkyl chain ranges from 0.001 to 1, more preferably from 0.005 to 0.5, and better still from 0.01 to 0.1.

Preferably, the weight ratio between the total amount of thickening polymer(s) and the total amount of alcohols comprising a C1-C8 alkyl chain ranges from 0.001 to 1, more preferably from 0.005 to 0.5, better still from 0.01 to 0.1.

### Water

The composition used according to the present invention also comprises water, which preferably represents from 40 to 95% by weight, more preferably from 50 to 90% by weight, better still from 55 to 85% by weight, and even more preferably from 65 to 80% by weight, relative to the total weight of the composition.

### Additives

The cosmetic composition used according to the present invention may further comprise one or more additive(s) other than the compounds of the invention.

As additives that may be used in accordance with the invention, mention may be made of fatty substances; cationic, anionic or amphoteric surfactants or mixtures thereof; antidandruff agents; anti-seborrhea agents; agents for preventing hair loss and/or for promoting hair regrowth; vitamins and provitamins including panthenol; sunscreens; mineral or organic pigments; sequestrants; plasticizers; solubilizers; acidifying agents; opacifiers or nacreous agents; antioxidants; hydroxy acids; fragrances and preserving agents.

Needless to say, a person skilled in the art will take care to select this or these optional additional compound(s) such that the advantageous properties intrinsically associated with the composition according to the invention are not, or are not substantially, adversely affected by the envisaged addition(s).

The above additives may generally be present in an amount, for each of them, of between 0.001 and 20% by weight, relative to the total weight of the composition.

According to a preferred embodiment of the present invention, the composition may comprise one or more anionic polymers, different from thickening polymers described previously, preferably selected from hyaluronic acid and derivatives thereof.

Hyaluronic acid can be represented by the following chemical formula:

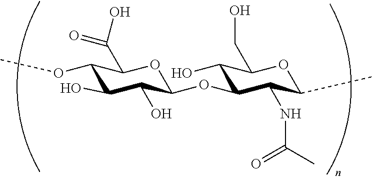

In the context of the present invention, the term “hyaluronic acid” covers in particular the basic unit of hyaluronic acid of formula:

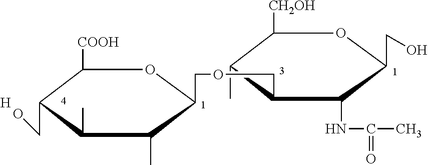

This is the smallest fraction of hyaluronic acid comprising a disaccharide dimer, namely D-glucuronic acid and N-acetylglucosamine.

The term “hyaluronic acid and derivatives thereof” also comprises, in the context of the present invention, the linear polymer comprising the polymeric unit described above, linked together in the chain via alternating β(1,4) and (1,3) glycosidic linkages, having preferably a molecular weight (Mw) that can range between 380 and 13,000,000 daltons. This molecular weight depends in large part on the source from which the hyaluronic acid is obtained and/or on the preparation methods.

The term “hyaluronic acid and derivatives thereof” also comprises, in the context of the present invention, hyaluronic acid salts. As the salts, mention may be made of alkaline metal salts such as sodium salts and potassium salts, alkaline earth metal salts such as magnesium salts, ammonium salts, and mixtures thereof.

Preferably the composition used according to the invention comprises hyaluronic acid and/or a salt thereof, preferably hyaluronic acid and/or an alkaline metal or alkaline earth metal salt thereof; and more preferably sodium hyaluronate.

The total amount of the anionic polymer(s), different from thickening polymers, in the composition according to the present invention may be from 0.001 to 0.1% by weight, preferably from 0.002 to 0.05% by weight, and more preferably from 0.005 to 0.02% by weight, relative to the total weight of the composition.

The total amount of the hyaluronic acid and derivatives thereof in the composition according to the invention may be from 0.001 to 0.1% by weight, preferably from 0.002 to 0.05% by weight, and more preferably from 0.005 to 0.02% by weight, relative to the total weight of the composition.

Preferably, the composition according to the invention is provided in the form of a thickened hydroalcoholic lotion or composition, and more preferably in the form of a hydroalcoholic gelified lotion or composition.

Preferably, the composition used in the process according to the invention comprises:
(i) one or more thickening polymers, chosen from crosslinked (meth)acrylic acid homopolymers, preferably crosslinked by an allyl ether of an alcohol of the sugar series; and/or crosslinked copolymers of (meth)acrylic acid and of (C1-C6)alkyl acrylate; preferably crosslinked copolymers of (meth)acrylic acid and of ethyl acrylate; preferably present in the composition in an amount ranging from 0.01 to 5% by weight, more preferably from 0.02 to 1% by weight, and better still from 0.05 to 0.5% by weight, relative to the total weight of the composition;
(ii) one or more compounds of formula (I) chosen from menthol, menthoxy propanediol, and mixture thereof;
preferably present in the composition in an amount ranging from 0.05 to 5% by weight, more preferably from 0.1 to 1% by weight, relative to the total weight of the composition;
(iii) one or more oxyalkylenated C8-C30 fatty alcohols, chosen from oxyethylenated fatty alcohols comprising a C₁₀-C₁₈ alkyl chain and from 5 to 20 oxyethylene units, and more preferably from oxyethylenated fatty alcohols comprising a C₁₀-C₁₈ alkyl chain and from 10 to 15 of oxyethylene units;
preferably present in the composition in an amount ranging from 0.1 to 10% by weight, preferably from 0.2 to 5% by weight, more preferably from 0.5 to 3% by weight, relative to the total weight of the composition;
(iv) one or more alcohols comprising a C1-C8 alkyl chain, chosen from monoalcohols comprising from 2 to 8 carbon atoms, diols comprising from 2 to 8 carbon atoms and mixtures thereof, and more preferably from monoalcohols comprising from 2 to 8 carbon atoms, ethanol being most preferred;
preferably present in the composition according to the invention in an amount ranging from 10 to 50% by weight, more preferably from 15 to 40% by weight, most preferably from 18 to 35% by weight, relative to the total weight of the composition; and
(v) water, preferably present in an amount ranging from 40 to 95% by weight, more preferably from 50 to 90% by weight, better still from 55 to 85% by weight, and even more preferably from 65 to 80% by weight, relative to the total weight of the composition.

The following example serves to illustrate the invention without, however, being limiting in nature.

In the examples that follow and unless otherwise indicated, the amounts are given as mass percentages of active material relative to the total weight of the composition (% AM=% active matter).

### EXAMPLE 1

The following composition according to the invention was prepared:

| | Composition |
|---|---|
| | (% AM) |
| PEG-60 Hydrogenated | 0.25 |
| castor oil | |
| Carbomer | 0.3 |
| Polysorbate-21 | 0.5 |
| L-menthol | 0.3 |
| Laureth-12 | 1 |
| Sodium hyaluronate | 0.01 |
| Ethanol | 25 |
| Water | Qs 100% |

A study was done on 18 braided hair women aged 20-40 years, having their braids on for a minimum of 2 weeks. The composition was applied on scalp and braided hair at least 3 times during a period of 7 days.

The study shows that the product provided immediate and long-lasting relief of itchiness, dandruff and discomfort; pleasant and soothing sensation on the scalp. It also shows that braided hair and scalp are moisturized and nourished. The composition makes the braided hair texture soft.

1-15. (canceled)

16. A method for treating braided hair, comprising applying to the braided hair a cosmetic composition comprising:
(i) at least one thickening polymer;
(ii) at least one compound of formula (I): 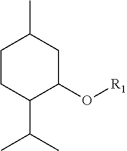
wherein R1 represents a hydrogen atom or a linear or branched, saturated or unsaturated, C1-C12 alkyl radical, optionally substituted by one or more OH groups and/or one or more NRR′ groups with R and R′ independently chosen from a hydrogen atom or a linear or branched, saturated or unsaturated, C1-C4 alkyl radical;
(iii) at least one oxyalkylenated C8-C30 fatty alcohol;
(iv) at least one alcohol comprising a C1-C8 alkyl chain, and
(v) water.

17. The method of claim 16, wherein the at least one thickening polymer is chosen from thickening polymers comprising acrylic and/or methacrylic units.

18. The method of claim 16, wherein the at least one thickening polymer is chosen from:
(a) (meth)acrylic associative thickeners;
(b) cross-linked (meth)acrylic acid homopolymers and copolymers;
(c) copolymers of (meth)acrylic acid and of (C1-C6)alkyl (meth)acrylate;
(d) non-ionic homopolymers and copolymers comprising ethylenically unsaturated monomer of ester and/or amide type;
(e) at least one of ammonium (meth)acrylate homopolymer or copolymer of ammonium (meth)acrylate and of (meth)acrylamide;
(f) (meth)acrylamido(C₁-C₄)alkylsulfonic acid homopolymers and copolymers;
(g) cross-linked (meth)acryloyloxy(C₁-C₄)alkyltri(C₁-C₄)alkylammonium homopolymers and copolymers; or (h) mixtures of two or more thereof.

19. The method of claim 16, wherein the at least one thickening polymer is chosen from:
cross-linked (meth)acrylic acid homopolymers;
cross-linked copolymers of (meth)acrylic acid and of (C1-C6)alkyl acrylate; or combinations of two or more thereof.

20. The method of claim 16, wherein the total amount of thickening polymer(s) ranges from 0.01% to 5% by weight, relative to the total weight of the composition.

21. The method of claim 16, wherein the total amount of thickening polymer(s) ranges from 0.05% to 5% by weight, relative to the total weight of the composition.

22. The method of claim 16, wherein in formula (I), R1 represents a hydrogen atom or a linear or branched, saturated or unsaturated, C1-C6 alkyl radical, substituted by one or more hydroxyl groups.

23. The method of claim 16, wherein the compound of formula (I) is chosen from menthol, menthoxy propanediol, or mixture thereof.

24. The method of claim 16, wherein the total amount of compound(s) of formula (I) ranges from 0.05% to 5% by weight, relative to the total weight of the composition.

25. The method of claim 16, wherein the at least one oxyalkylenated C₈-C₃₀ fatty alcohol is chosen from oxyethylenated fatty alcohols comprising a C₁₀-C₁₈ alkyl chain and from 10 to 15 oxyethylene units.

26. The method of claim 16, wherein the at least one oxyalkylenated C₈-C₃₀ fatty alcohol is chosen from oxyethylenated lauryl alcohol comprising 12 units of ethylene oxide (laureth-12).

27. The method of claim 16, wherein the total amount of oxyalkylenated C₈-C₃₀ fatty alcohol(s) ranges from 0.1% to 10% by weight, relative to the total weight of the composition.

28. The method of claim 16, wherein the composition comprises at least one additional non-ionic surfactant different from the at least one oxyalkylenated C₈-C₃₀ fatty alcohol in a total amount ranging from 0.05 to 5% by weight, relative to the total weight of the composition.

29. The method of claim 28, wherein the at least one additional non-ionic surfactant different from the at least one oxyalkylenated C8-C30 fatty alcohol is chosen from:
from 0.05% to 1% of saturated or unsaturated, oxyethylenated plant oils;
from 0.05% to 5% of esters of saturated or unsaturated, linear or branched, C8-C30 acids and of sorbitol;
or mixtures of two or more thereof,
wherein each amount is by weight, relative to the total weight of the composition.

30. The method of claim 16, wherein the total amount of alcohol(s) comprising a C₁-C₈ alkyl chain ranges from 10 to 50% by weight, relative to the total weight of the composition.

31. The method of claim 16, wherein the total amount of water ranges from 40 to 95% by weight, relative to the total weight of the composition.

32. The method of claim 16, further comprising at least one anionic polymer chosen from hyaluronic acid or derivatives thereof.

33. The method of claim 16, wherein the composition improves moisturization and/or softness of the braided hair.

34. A cosmetic composition comprising:
(i) at least one thickening polymer;
(ii) at least one compound of formula (I) 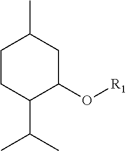
wherein R1 represents a hydrogen atom or a linear or branched, saturated or unsaturated, C1-C12 alkyl radical, optionally substituted by one or more OH groups and/or one or more NRR′ groups with R and R′ independently chosen from a hydrogen atom or a linear or branched, saturated or unsaturated, C1-C4 alkyl radical;
(iii) at least one oxyalkylenated C8-C30 fatty alcohol;
(iv) at least one alcohol comprising a C1-C8 alkyl chain, and
(v) water.

35. A cosmetic composition comprising:
(i) from 0.01% to 5% of at least one thickening polymer;
(ii) from 0.05% to 5% of at least one compound of formula (I) 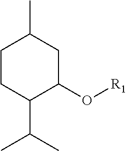
wherein R1 represents a hydrogen atom or a linear or branched, saturated or unsaturated, C1-C12 alkyl radical, optionally substituted by one or more OH groups and/or one or more NRR′ groups with R and R′ independently chosen from a hydrogen atom or a linear or branched, saturated or unsaturated, C1-C4 alkyl radical;
(iii) at least one oxyalkylenated C8-C30 fatty alcohol;
(iv) at least one alcohol comprising a C1-C8 alkyl chain, and
(v) water,
wherein each amount is by weight, relative to the total weight of the composition.
